SEQUENCE LISTING (10 sequences)

SEQ ID NO: 1 - protein, 320 aa, Homo sapiens
  MISC_FEATURE (268)..(305): M9 domain
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGSNFGGGGSYNDFGNYNNQSSNFGPMKGGNFGGRSSGPYGGGGQYFAKPRNQGGYGGSSSSSSYGSGRRF

SEQ ID NO: 2 - protein, 12 aa, Homo sapiens
GQYFAKPRNQGG

SEQ ID NO: 3 - protein, 12 aa, Homo sapiens
SSQRGRSGSGNF

SEQ ID NO: 4 - protein, 315 aa, Homo sapiens
  MISC_FEATURE (268)..(305): M9 domain
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPDAHLTVKKIFVGGIKEDTEEHHLRDYFEQYGKIEVIEIMTDRGSGKKRGFAFVTFDDHDSVDKIVIQKYHTVNGRNCEVRKALSKQEMASASSSQRGRSGSGNFGGGRGGGFGGNDNFGRGGNFSGRGGFGGSRGGGGYGGSGDGYNGLGNDGSNFGGGGSYNDFGNYNNQSSNFGPMKGGNFEGRSSGPHGGGGQYFAKPRNQGGYGGSSSSSSYG

SEQ ID NO: 5 - protein, 317 aa, Homo sapiens
  MISC_FEATURE (268)..(302): M9 domain
MSKSESPKEPKQLRKLFIGGLSFETTNESLRSHFEQWGTLMDCVVMRDPNTKCSRGFGFVTYATVEEVDAAMNARPHKVDGRVVESKRAVSREDSQRPGAHLTVKKIFVGGIKEDTKEHHLRDYFEQYGKIEVIEIMTDRGSGKKRGFAFVTFDDHDSVDKIVIQKYHTVNGHICEVRKALSKQEMASTSSSQRGQSGSGNFSGGRGGGFSGNDNFGHGGNFSGRGGFGGSRGAGGYGGSGDGYNGFGNDGSNFGGGGSYNDFGNYNYQSSNFRPMKGGNFGGRSSGPYGGGGNTLQNQGGYGSSSSSSSYGSGRRF

SEQ ID NO: 6 - protein, 320 aa, Homo sapiens
  MISC_FEATURE (268)..(305): M9 domain
MSKSESPKEPEQLRKLFIGGLSFETTDESLRSHFEQWGMLTDCVVMRDPNTKHPRGFGFVTYATVEEVDAAMNARPHKVDGRVVEPKRAVSREDSERPGAHLTVKKIFVGGIKEDTEEHHLRDYFGQYGKIEVIEIMTDRGSGKKRGFAFVTFDDHDSVDKIVIQKYHTVNGHNCEVRKALSKQEMASASSSQRGRSGSGNFGGGRGGGFGGNDNFGHGGNFSGRGGFGGSRAGGRYGGSGDGSNGFGDDGSNFGGGGSYNDFGNYNNQSSNFGPMKGGNFGGRNSGPYGGGGQYFAKPRNQGGYGSSSSSSSYGSGRRF

SEQ ID NO: 7 - protein, 321 aa, Homo sapiens
  MISC_FEATURE (268)..(305): M9 domain
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKILVGGIKEGTEEHHLRDYFEQYGKIEVIEIMTDRGSGKKRGFAFVTFDDHDSVDKTVIQKYHTVNGHNCEVRKALSKQEMASASSSQRGRSGSGNFGGGRGGGFGGNDNFGRGGNFSGRGGFGGSHGGGGYGGSGDGYNGFGNDGSNFGGGGSYNDFGNYNNQSSNFGPMKGGNFGGRSSGPYGGGGQYFAKPRNQVAMAVPAAAVAMAVAEDFN

SEQ ID NO: 8 - protein, 320 aa, Homo sapiens
  MISC_FEATURE (268)..(305): M9 domain
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPDAHLTVKKIFVGGIKEDTEEHHLRDYFEQYGKIEVIEIMTDRGGGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGSNFGGGGSYDDFGNYNNQSSNFGPMKGGNFEGRSSGPHGGGGQYFAKPRNQGGYGGSSSSSSYGSGRRF

SEQ ID NO: 9 - protein, 320 aa, Homo sapiens
  MISC_FEATURE (268)..(305): M9 domain
MSKSESPKEPEQLRKLFIGGLSFETTDESLRSHFAQWGTLTDCVVMRDPNTKRSRGFGFVTYATVEEVDAAMNARSHKVDGRVVEPKRAVSREDSQRPGAHLTVKKIFVGGVKEDTEEHHLRDYFEQYGKIEVVEIMTDRGSGKKRGFAFVTFDDHDSVDKTVIQKYHTVNGHNCEVRKALSKQEMASASSSQRGRSGSGNFGGGRGGGFGGNGNFGRGGNFSGRGGFGGSHGGGGYGGSGDGYNGFGNDGSNFGGGGSYNDFGNYNNQSSNFGPMKGGNFGGRSSGPYGGGGQYFAKPRNQVAMAVPAAAVAMAVAEDF

SEQ ID NO: 10 - protein, 372 aa, Homo sapiens
MSKSESPKEPEQLRKLFIGGLSFETTDESLRSHFEQWGTLTDCVVMRDPNTKRSRGFGFVTYATVEEVDAAMNARPHKVDGRVVEPKRAVSREDSQRPGAHLTVKKIFVGGIKEDTEEHHLRDYFEQYGKIEVIEIMTDRGSGKKRGFAFVTFDDHDSVDKIVIQKYHTVNGHNCEVRKALSKQEMASASSSQRGRSGSGNFGGGRGGGFGGNDNFGRGGNFSGRGGFGGSRGGGGYGGSGDGYNGFGNDGGYGGGGPGYSGGSRGYGSGGQGYGNQGSGYGGSGSYDSYNNGGGGGFGGGSGSNFGGGGSYNDFGNYNNQSSNFGPMKGGNFGGRSSGPYGGGGQYFAKPRNQGGYGGSSSSSSYGSGRRF

(19) United States
(12) Patent Application Publication
(10) Pub. No.: US 2015/0192580 A1
(43) Pub. Date: Jul. 9, 2015

(54) BIOMARKER FOR NEURODEGENERATION IN NEUROLOGICAL DISEASE

(71) Applicants: MICHAEL C. LEVIN, MEMPHIS, TN (US); SANGMIN LEE, CORDOVA, TN (US)

(72) Inventors: MICHAEL C. LEVIN, MEMPHIS, TN (US); SANGMIN LEE, CORDOVA, TN (US)

(21) Appl. No.: 14/665,050

(22) Filed: Mar. 23, 2015

Related U.S. Application Data
(63) Continuation of application No. 13/393,900, filed on May 10, 2012.
(60) Provisional application No. 61/239,592, filed on Sep. 3, 2009.

(51) Int. Cl. G01N 33/564; C07K 14/47

(52) U.S. Cl. CPC G01N 33/564; C07K 14/4713; G01N 2800/2814; G01N 2800/2821; G01N 2800/2828; A61K 39/00

(57) ABSTRACT

A method of detecting or diagnosing a neurodegenerative disease in a subject by an immunoassay is provided. The immunoassay comprises obtaining a sample from a subject and assaying the sample for the presence of autoantibodies against certain epitopes of heterogeneous nuclear ribonuclear protein A1 (hnRNP A1). The presence of antibodies against a polypeptide or a fragment of polypeptide having the sequence of SEQ ID NO: 1, 4, 5, 6, 7, 8, 9 or 10, an epitope within the M9 shuttling sequence and/or an epitope within the RGG domain of hnRNP A1 indicate a neurodegenerative disease in the subject. Methods of treating neurodegenerative diseases are also provided. In one embodiment, the method of treating a neurodegenerative disease comprises administering, to a subject, a composition comprising hnRNP A1, fragments of the polypeptide and/or a polypeptide comprising epitopes within the M9 and/or RGG domains of hnRNP A1.

### CROSS-REFERENCE TO RELATED APPLICATIONS

This application is a continuation of U.S. Ser. No. 13/393,900, filed May 10, 2012, which is the U.S. national stage application of International Patent Application No. PCT/US2010/047690, filed Sep. 2, 2010, which claims the benefit of U.S. Provisional Application Ser. No. 61/239,592, filed Sep. 3, 2009, the disclosures of which are hereby incorporated by reference in their entireties, including all figures, tables and amino acid or nucleic acid sequences.

The Sequence Listing for this application is labeled “Seq-List.txt” which was created on Sep. 2, 2010 and is 24 KB. The entire contents of the sequence listing is incorporated herein by reference in its entirety.

### SUMMARY OF THE INVENTION

Aspects of the present invention provide a method of detecting or diagnosing a neurodegenerative disease or condition in a subject. Further aspects of the invention provide a method of monitoring a subject over a period of time to detect the development or progress of a neurodegenerative disease or condition. In various embodiments, a method of detecting or diagnosing a neurodegenerative disease or condition in a subject is performed by obtaining a biological sample from a subject and assaying the sample to determine the presence or absence of autoantibody in said sample, wherein the presence or elevated presence of autoantibodies correlates with a neurodegenerative disease or condition in said subject. The autoantibodies bind to heterogeneous nuclear ribonuclear protein A1 (hnRNPA1; SEQ ID NO: 1, 4, 5, 6, 7, 8, 9 or 10) or to, for example, a polypeptide fragment comprising a consecutive span of SEQ ID NO: 1, 4, 5, 6, 7, 8, 9 or 10 that includes an epitope within the M9 shuttling sequence (e.g., amino acids 293-304 of hnRNP A1 (SEQ ID NO: 1; GQYFAKPRNQGG; SEQ ID NO: 2) within the M9 shuttling sequence and/or an epitope within the RGG domain (e.g., amino acids 191-202 of hnRNP A1 (SEQ ID NO: 1; SSQRGRSGSGNF; SEQ ID NO: 3) within the RGG domain.

Other aspects of the invention provide for methods of treating neurodegenerative diseases or conditions characterized by the presence of autoantibodies that specifically bind to hnRNP A1 and/or epitopes within the M9 and/or RGG domains of the hnRNP A1 polypeptide, for example. These methods comprise the administration of a composition comprising hnRNP A1, fragments of the polypeptide and/or a polypeptide comprising (or consisting of) epitopes within the M9 and/or RGG domains of hnRNP A1 to a subject having a neurodegenerative disease or condition.

### BRIEF DESCRIPTION OF THE FIGURES

FIG. 1: hnRNP A1 RNA splice-variants in the neurons (SEQ ID NO: 1 (original), SEQ ID NO: 4 (clone 2), SEQ ID NO: 5 (clone 2), SEQ ID NO: 6 (clone 3), SEQ ID NO: 7 (clone 4), SEQ ID NO: 8 (clone 5), SEQ ID NO: 9 (clone 6)). The RGG domain (amino acids 191-253) and M9 shuttling domain (amino acids 268-305) are indicated by underlining.

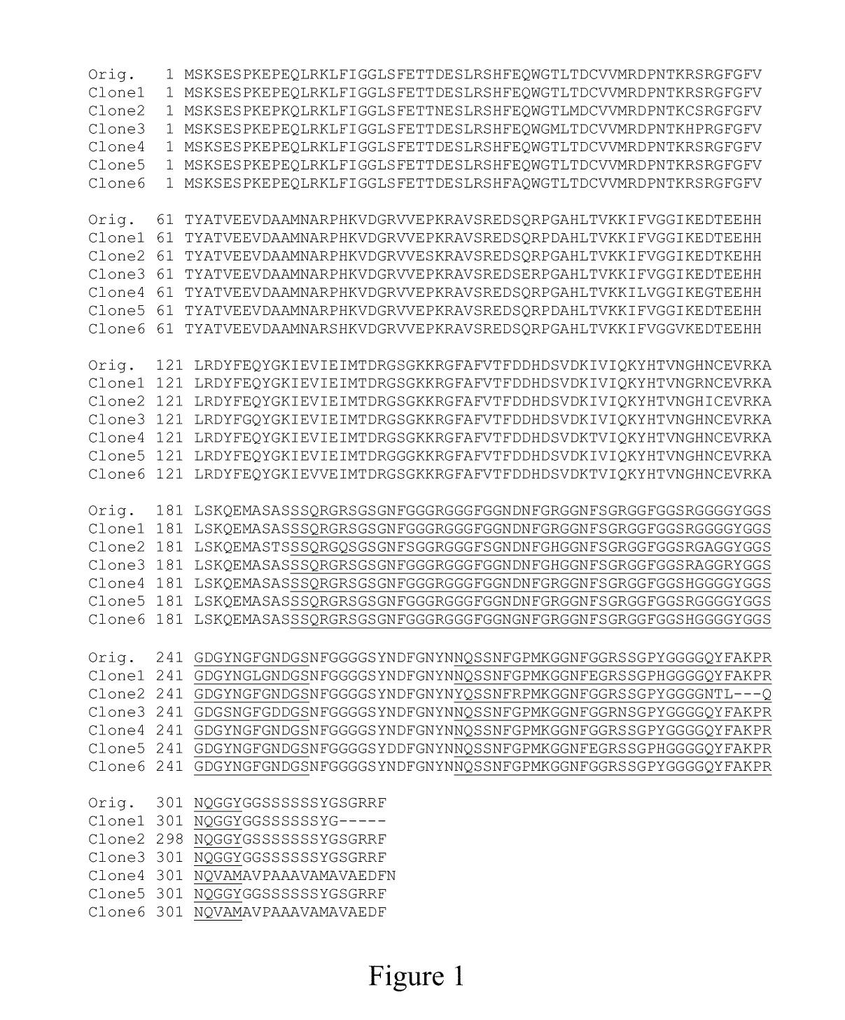

FIG. 2: Reactivity by Western blot of antibodies purified from the sera from patients with multiple sclerosis (MS) and tropical spastic paraparesis (TSP) with the hnRNP A1-M9 target epitope (AA²⁹³⁻³⁰⁴; SEQ ID NO: 2). These western blots using M9 as the protein antigen show that all of the antibodies purified from the sera from MS patients reacted with M9, as did cerebrospinal fluid (CSF) samples from these patients. TSP patients were used as positive controls. There was no reactivity with antibodies isolated from normal controls.

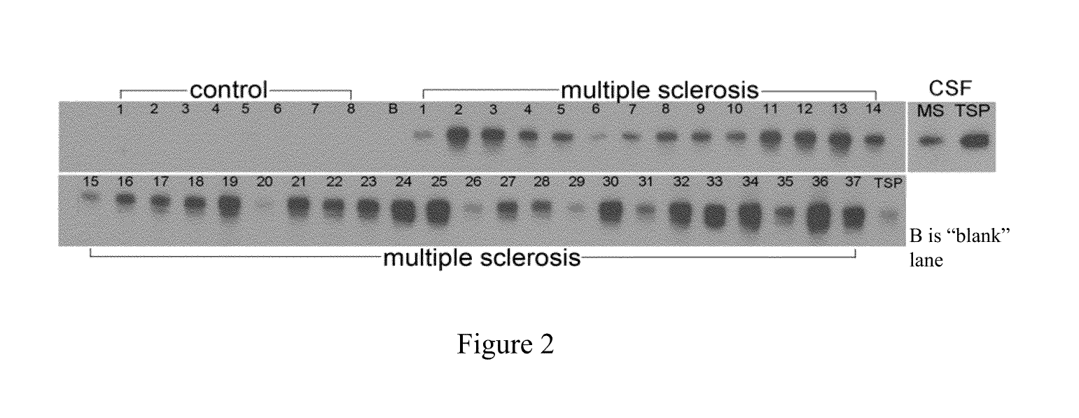

### DETAILED DESCRIPTION OF THE INVENTION

Before the present compositions and methods for screening, monitoring, and treating neurological disorders and neurodegenerative syndromes are disclosed and described, it is to be understood that this invention is not limited to the particular process steps and materials disclosed herein as such process steps and materials may vary somewhat. It is also to be understood that the terminology employed herein is used for the purpose of describing particular embodiments only and is not intended to be limiting since the scope of the present invention will be limited only by the appended claims and equivalents thereof.

It must also be noted that, as used in this specification and the appended claims, the singular forms “a”, “an” and “the” include plural referents unless the content clearly dictates otherwise. Additionally, the terms “comprising”, “consisting of” and “consisting essentially of” are defined according to their standard meaning. The terms may be substituted for one another throughout the instant application in order to attach the specific meaning associated with each term.

In describing and claiming the present invention, the following terminology will be used in accordance with the definitions set out below.

As used herein, “autoantibody” means an antibody produced by the immune system of a subject that is directed to, and specifically binds to, a naturally occurring protein or tissue in the subject. The term “specifically binds to” means that an antibody can bind preferably in a competitive binding assay to its binding partner, in this case hnRNP A1, as assessed using either recombinant forms of the protein, epitopes therein (e.g., epitopes within the M9 and/or RGG domains), or native proteins present on the surface of isolated target cells. Competitive binding assays and other methods for determining specific binding are further described below and are well known in the art.

As used herein, “biological sample” means a sample of biological material taken from a subject for performing an assay and determining whether autoantibodies against hnRNPA1 and/or an epitope within the M9 and/or RGG domains of the hnRNP A1 polypeptide are present in the subject's body and/or the quantity or concentration of such autoantibodies. Exemplary biological samples are biological fluids, such as blood, blood plasma, cerebrospinal fluid, and the like.

The term “subject” includes mammals such as, but not limited to, apes, chimpanzees, orangutans, humans, monkeys, dogs, cats, guinea pigs, mice and rats.

As used herein, an “assay” means any assay that determines the physical presence of autoantibodies in a biological sample. Non-limiting examples of assays that can be used for making qualitative and quantitative measurements of autoantibodies include immunoblot, ELISA, sandwich ELISA, competitive peptide ELISA assays, immunocytochemistry of hnRNPA1 containing cells and tissues (e.g., brain, nerves, etc.), dot blots, pin assays, immunoprecipitations, radioimmunoassays and the like.

As used herein, “effective amount” means an amount capable of producing a selected effect. Thus, an effective amount of a peptide capable of binding hnRNP A1 autoantibodies and treating neurodegenerative disease is an amount that produces this effect and renders hnRNPA1 specific antibodies incapable of specifically binding the polypeptide.

As used herein, “peptide” means peptides of any length and includes proteins. The terms “polypeptide” and “oligopeptide” are used herein without any particular intended size limitation, unless a particular size is otherwise stated. In certain aspects of the invention, fragments of SEQ ID NO: 1, 4, 5, 6, 7, 8, 9 or 10 are used for treatment of neurodegenerative diseases and diagnosis or detecting a neurodegenerative disease in a subject.

“Peptide fragments”, “polypeptide fragments” and/or “epitopes” of SEQ ID NO: 1, 6, 8 or 9 comprise a span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318 or 319 consecutive amino acids of SEQ ID NO: 1, 6, 8 or 9, provided that this span of consecutive amino acids also includes SEQ ID NO: 2 and/or SEQ ID NO: 3 or a span of 5, 6, 7, 8, 9, 10 or 11 contiguous amino acids of SEQ ID NO: 2 and/or 3.

“Peptide fragments”, “polypeptide fragments” and/or “epitopes” of SEQ ID NO: 4 comprise a span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313 or 314 consecutive amino acids of SEQ ID NO: 4, provided that this span of consecutive amino acids also includes SEQ ID NO: 2 and/or SEQ ID NO: 3 or a span of 5, 6, 7, 8, 9, 10 or 11 contiguous amino acids of SEQ ID NO: 2 and/or 3.

“Peptide fragments”, “polypeptide fragments” and/or “epitopes” of SEQ ID NO: 5 comprise a span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315 or 316 consecutive amino acids of SEQ ID NO: 5, provided that this span of consecutive amino acids also includes SEQ ID NO: 2 and/or SEQ ID NO: 3 or a span of 5, 6, 7, 8, 9, 10 or 11 contiguous amino acids of SEQ ID NO: 2 and/or 3.

“Peptide fragments”, “polypeptide fragments” and/or “epitopes” of SEQ ID NO: 7 comprise a span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319 or 320 consecutive amino acids of SEQ ID NO: 7, provided that this span of consecutive amino acids also includes SEQ ID NO: 2 and/or SEQ ID NO: 3 or a span of 5, 6, 7, 8, 9, 10 or 11 contiguous amino acids of SEQ ID NO: 2 and/or 3.

“Peptide fragments”, “polypeptide fragments” and/or “epitopes” of SEQ ID NO: 10 comprise a span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371 consecutive amino acids of SEQ ID NO: 10, provided that this span of consecutive amino acids also includes SEQ ID NO: 2 and/or SEQ ID NO: 3 or a span of 5, 6, 7, 8, 9, 10 or 11 contiguous amino acids of SEQ ID NO: 2 and/or 3. SEQ ID NO: 2 is found at positions 345-366 in SEQ ID NO: 10.

It should be understood that the phrases “comprising SEQ ID NO: 2”, “comprising SEQ ID NO: 3”, “comprising a contiguous span of SEQ ID NO: X”, where X is 1, 4, 5, 6, 7, 8, 9 or 10, “comprising a fragment of SEQ ID NO: X”, where X is 1, 4, 5, 6, 7, 8, 9 or 10 indicate a polypeptide fragment that has fewer amino acids than the naturally occurring hnRNP A1 polypeptide (i.e., SEQ ID NO: 1, 4, 5, 6, 7, 8, 9 or 10). In various aspects of the invention, a contiguous span of amino acids that includes SEQ ID NO: 2 and 3 or fragments of SEQ ID NO: 2 or (e.g., fragments of 5 to 11 contiguous amino acids of SEQ ID NO: or fragments of SEQ ID NO: 3 containing 5 to 11 contiguous amino acids of SEQ ID NO: 3), can be used in the diagnostic and therapeutic protocols disclosed herein.

In some aspects of the invention, polypeptide fragments can be fused (operably linked) to heterologous sequences, such as affinity tags, immunoglobulin constant regions (heavy chain or light chain constant regions). The heterologous sequence may be located upstream (N-ter) or downstream (C-ter) from the sequence of the polypeptide fragments disclosed herein and may comprise any functional region, providing, for instance, increased stability, targeting or bioavailability of the fusion protein; facilitating purification or production, or conferring on the molecule additional biological activity. Specific examples of such heterologous sequences include a GST sequence, a His tag sequence, a multimerication domain, the constant region of an immunoglobulin molecule. The term “fused” or “operably linked” indicates that the polypeptide and additional amino acid domain are associated through peptide linkage, either directly or via spacer residues. In this manner, the fusion protein can be produced recombinantly, by direct expression in a host cell of a nucleic acid molecule encoding the same, as will be discussed below. Also, if needed, the additional amino acid sequence included in the fusion proteins may be eliminated, either at the end of the production/purification process or in vivo, e.g., by means of an appropriate endo-/exopeptidase. For example, a spacer sequence included in the fusion protein may comprise a recognition site for an endopeptidase (such as a caspase) that can be used to separate by enzymatic cleavage the desired polypeptide variant from the additional amino acid domain, either in vivo or in vitro.

As used herein, a “substantially homologous polypeptide” or “substantially homologous peptide” means a polypeptide or peptide that retains the functionality of binding hnRNP A1 specific autoantibodies or autoantibodies specific to an epitope within the M9 and/or RGG domains of the hnRNP A1 polypeptide. In some aspects of the invention, such polypeptides and peptides block the ability of hnRNP A1 autoantibodies to mediate or cause neurodegenerative diseases or disorders.

A substitution variant is one that contains a conservative substitution of one or more amino acid residues. A conservative substitution is a substitution of one amino acid for another wherein functionality of the peptide is retained. Amino acid residues belonging to certain conservative substitution groups can sometimes substitute for another amino acid residue in the same group. Substitution groups have been variously defined, however, one such definition is as follows: Pro; Ala, Gly; Ser, Thr; Asn, Gln; Asp, Glu; His; Lys, Arg; Cys; Ile, Leu, Met, Val; and Phe, Tip, Tyr.

Aspects of the present invention provide a method of detecting or diagnosing a neurodegenerative disease or condition in a subject. Further aspects of the invention provide a method of monitoring a subject over a period of time to detect the development or progress of a neurodegenerative disease or condition. In various embodiments, a method of detecting or diagnosing a neurodegenerative disease or condition in a subject is performed by obtaining a biological sample from a subject and assaying the sample to determine the presence or absence of autoantibody in said sample, wherein the presence or elevated presence of autoantibodies correlates with a neurodegenerative disease or condition in said subject. The autoantibodies bind to heterogeneous nuclear ribonuclear protein A1 (hnRNPA1; SEQ ID NO: 1) or to, for example, an M9 epitope of hnRNP A1 (SEQ ID NO: 2). In various aspects of the invention, the subject has not been exposed to HTLV-1 and/or does not have tropical spastic paraparesis.

According to one embodiment, a neurodegenerative disease or condition is diagnosed by obtaining a biological sample from a subject and assaying the sample for the presence of autoantibodies specific for hnRNP A1 or epitopes thereof. Some embodiments in this aspect of the invention measure levels of autoantibodies with and compare such levels to a baseline (control) value. For example, the baseline value may be a level of autoantibodies previously obtained from a sample from the subject at a time when the subject did not have symptoms of a neurodegenerative disease or the baseline value may be an average or mean value of autoantibodies in a population of control individuals (individuals not having a neurodegenerative disease).

Another aspect of the invention provides methods of monitoring the progression of disease in a subject. In various embodiments of this aspect of the invention, the subject may be treated with a medication (subjected to a therapeutic protocol) and the progression or therapeutic benefit of the medication (therapeutic protocol) is assessed by measurement of autoantibodies in biological samples obtained from the individual. Thus, in this aspect of the invention, biological samples from a subject are assayed for autoantibodies specific for hnRNP A1 and/or epitopes thereof before a therapeutic protocol is administered to the subject (base line value) and at some later point in time. As described above, levels of autoantibodies from the subject are measured and then compared to autoantibody levels designated as the baseline value (e.g., the level of autoantibodies in a biological sample from the individual before onset of disease or before subject has undergone a therapeutic regimen). Time between the start of a treatment regimen or onset of a disease can be weeks, months or years, depending on the condition or disease. In various aspects of the invention, the subject has not been exposed to HTLV-1 and/or does not have tropical spastic paraparesis.

With respect to biological samples, the samples can be obtained by various methods known in the art. For example, a sample of cerebral spinal fluid (CSF) may be obtained by any method known in the art for obtaining a sample of cerebral spinal fluid from a subject including, for example, by a spinal tap (typically, lumbar puncture). Blood and blood serum samples can be obtained by, for example, venipuncture.

A neurodegenerative disease or condition includes Huntington's disease, Parkinson's disease, post-traumatic stress disorder, stroke, spinal cord trauma, traumatic brain injury, multi-infarct dementia, epilepsy, amyotrophic lateral sclerosis, viral induced dementia (for example, AIDS induced dementia), neurodegeneration associated with bacterial infection, brain ischemia, multiple sclerosis, Alzheimer's disease, senile dementia of the Alzheimer's type, mild cognitive impairment, age-related cognitive decline, corticobasal degeneration, dementia pugilistica, Down's syndrome, myotonic dystrophy, Niemann-Pick disease, Pick's disease, lower lateral sclerosis, paraneoplastic syndromes, encephalopathy, vascular dementia, primary progressive aphasia, diffuse Lewy body disease, progressive supranuclear palsy, Jacob Cruetzfeldt disease, Gerstmann Straussler disease, hydrocephalus, hereditary spinal paraplegia, myasthenia gravis, peripheral neuropathy, striatonigral degeneration, multi-system atrophy, familial tremor, Tourette's syndrome, myoclonus, Wilson's disease, Hallervorden-Spatz disease, neuroacanthocytosis, hemifacial spasm, Friedreich's ataxia, spinocerebellar ataxia, Sydenham's chorea, myositis and subacute sclerosing panencephalitis.

Other aspects of the invention provide for methods of treating neurodegenerative diseases or conditions characterized by the presence of autoantibodies to hnRNP A1 and/or epitopes within the M9 and/or RGG domains of the hnRNP A1 polypeptide. These methods comprise the administration of a composition comprising hnRNP A1, fragments of the polypeptide and/or a polypeptide comprising (or consisting of) the epitopes within the M9 and/or RGG domains of hnRNP A1 to a subject having a neurodegenerative disease or condition in an amount sufficient to neutralize or reduce the amount/level of hnRNP A1 specific antibody within the subject. Other embodiments provide for the administration of epitopes/peptides, such as SEQ ID NOs: 2 and/or 3 in an amount sufficient to bind to autoantibodies found in the CSF or vascular supply of a subject and block, inhibit or reduce the ability of such autoantibodies to bind hnRNP A1. In various embodiments of this aspect of the invention, the subject has not been exposed to HTLV-1 and/or does not have tropical spastic paraparesis.

As discussed above, neurodegenerative diseases or conditions suitable for treatment in this aspect of the invention include Huntington's disease, Parkinson's disease, post-traumatic stress disorder, stroke, spinal cord trauma, traumatic brain injury, multi-infarct dementia, epilepsy, amyotrophic lateral sclerosis, viral induced dementia (for example, AIDS induced dementia), neurodegeneration associated with bacterial infection, brain ischemia, multiple sclerosis, Alzheimer's disease, senile dementia of the Alzheimer's type, mild cognitive impairment, age-related cognitive decline, corticobasal degeneration, dementia pugilistica, Down's syndrome, myotonic dystrophy, Niemann-Pick disease, Pick's disease, lower lateral sclerosis, paraneoplastic syndromes, encephalopathy, vascular dementia, primary progressive aphasia, diffuse Lewy body disease, progressive supranuclear palsy, Jacob Cruetzfeldt disease, Gerstmann Straussler disease, hydrocephalus, hereditary spinal paraplegia, myasthenia gravis, peripheral neuropathy, striatonigral degeneration, multi-system atrophy, familial tremor, Tourette's syndrome, myoclonus, Wilson's disease, Hallervorden-Spatz disease, neuroacanthocytosis, hemifacial spasm, Friedreich's ataxia, spinocerebellar ataxia, Sydenham's chorea, myositis and subacute sclerosing panencephalitis.

In various embodiments, compositions comprising a pharmaceutically acceptable excipient and a peptide fragment comprising: a) SEQ ID NO: 2, optionally fused to a heterologous sequence; b) SEQ ID NO: 3, optionally fused to a heterologous sequence; or c) a peptide fragment of SEQ ID NO: 1, 4, 5, 6, 7, 8, 9 or 10. It is understood that the peptide fragment of SEQ ID NO: 1 comprising said contiguous span of amino acids is shorter than the full length of SEQ ID NO: 1.

In further embodiments, immunological kits for use in detecting autoantibodies against hnRNPA1 and/or an epitope within the M9 and/or RGG domains of the hnRNP A1 in biological samples are provided. Such kits can generally comprise one or more epitopes disclosed herein that can immunoreact with autoantibodies found in the biological sample. More specifically, the immunological kits can comprise, in suitable container(s), one or more autoantibody immunoreactive peptide fragments as disclosed herein. In some embodiments, the kits further comprise antibodies that bind to the peptide fragments and/or antibodies that bind to the human autoantibodies found in the biological sample). In certain embodiments, the antigen or can be provided bound to a solid support, such as for example a column matrix or well of a microtiter plate, a membrane (e.g., nitrocellulose, PVDF or similar material), beads, or dipsticks. Alternatively, the support can be provided as a separate element of the kit.

The immunological kits can also include detectable labels that are associated with, or linked to, the detecting antibody or to the antigen itself. Detectable labels that are associated with or attached to a secondary binding ligand are also contemplated. Such detectable labels include dyes, haptens, chemiluminescent or fluorescent molecules (rhodamine, fluorescein, green fluorescent protein, luciferase), biotin, radiolabels (³H, ³⁵S, ³²P, ¹⁴C, ¹³¹I, etc.) or enzymes (alkaline phosphatase, horseradish peroxidase).] The kits can further comprise suitable standards of predetermined amounts, including both antibodies and antigens. These can be used to prepare a standard curve for a detection assay.

The kits of the presently disclosed subject matter, regardless of type, can generally comprise one or more containers into which the biological agents are placed and suitably aliquoted. The components of the kits can be packaged either in aqueous media or in lyophilized form.

Accordingly, the following non-limiting embodiments are provided in the subject application:

1. A method of detecting or diagnosing a neurodegenerative disease or condition in a subject comprising:

a) obtaining a biological sample from the subject; and

b) assaying the sample for the presence of at least one autoantibody that binds to SEQ ID NO: 1, 4, 5, 6, 7, 8, 9 or 10, and wherein presence of said at least one autoantibody indicates that the subject has, or is at risk of developing, a neurodegenerative disease or condition,

wherein said assaying comprises:

i) contacting a polypeptide comprising SEQ ID NO: 1, 4, 5, 6, 7, 8, 9 or 10 with said biological sample and detecting the binding of autoantibodies, wherein said biological sample is a blood serum sample; or

ii) contacting an epitope comprising a peptide fragment of SEQ ID NO: 1, 4, 5, 6, 7, 8, 9 or 10 with a biological sample and detecting the binding of autoantibodies to said polypeptide fragment, wherein said peptide fragment comprises SEQ ID NO: 2, SEQ ID NO: 3 or both SEQ ID NO: 2 and SEQ ID NO: 3.

2. The method according to embodiment 1, wherein the autoantibody binds an epitope comprising:

a) SEQ ID NO: 2, optionally fused to a heterologous sequence;

b) SEQ ID NO: 3, optionally fused to a heterologous sequence; or

c) a peptide fragment of SEQ ID NO: 1, 4, 5, 6, 7, 8, 9 or 10, wherein said peptide fragment is a contiguous span of amino acids that contains SEQ ID NO: 2; SEQ ID NO: 3; both SEQ ID NO: 2 and SEQ ID NO: 3; or a span of 5, 6, 7, 8, 9, 10 or 11 contiguous amino acids of SEQ ID NO: 2 and/or 3, said contiguous span optionally being fused to a heterologous sequence.

3. The method according to embodiment 1, wherein the subject has a neurodegenerative disease or condition selected from Huntington's disease, Parkinson's disease, post-traumatic stress disorder, stroke, spinal cord trauma, traumatic brain injury, multi-infarct dementia, epilepsy, amyotrophic lateral sclerosis, viral induced dementia, AIDS induced dementia, neurodegeneration associated with bacterial infection, brain ischemia, multiple sclerosis, Alzheimer's disease, senile dementia of the Alzheimer's type, mild cognitive impairment, age-related cognitive decline, corticobasal degeneration, dementia pugilistica, Down's syndrome, myotonic dystrophy, Niemann-Pick disease, Pick's disease, lower lateral sclerosis, paraneoplastic syndromes, encephalopathy, vascular dementia, primary progressive aphasia, diffuse Lewy body disease, progressive supranuclear palsy, Jacob Cruetzfeldt disease, Gerstmann Straussler disease, hydrocephalus, hereditary spinal paraplegia, myasthenia gravis, peripheral neuropathy, striatonigral degeneration, multi-system atrophy, familial tremor, Tourette's syndrome, myoclonus, Wilson's disease, Hallervorden-Spatz disease, neuroacanthocytosis, hemifacial spasm, Friedreich's ataxia, spinocerebellar ataxia, Sydenham's chorea, myositis or subacute sclerosing panencephalistis.

4. The method according to embodiments 1-3, wherein the subject has a neurodegenerative disease or condition selected from Parkinson's disease, post-traumatic stress disorder, multiple sclerosis, Alzheimer's disease or senile dementia of the Alzheimer's type.

5. The method according to embodiments 1-4, wherein the subject has a family history of at least one neurodegenerative disease or condition.

6. The method according to embodiments 1-5, wherein said assay measures the level/amount of autoantibody in said sample, the level/amount of autoantibody is determined relative to a baseline value and wherein the baseline value is an average or mean value of the level/amount of autoantibody in blood serum samples from a population of control subjects.

7. A method of monitoring the development or progress of a neurodegenerative disease or condition in a subject over a period of time comprising conducting an assay according to any one of embodiments 1-6 at a first point in time and conducting said assay at a second point in time, said second point in time being later than said first point in time.

8. The method of embodiment 7, wherein said assay measures the level/amount of autoantibody in a sample from a subject after treatment for a neurodegenerative disease or condition, said first point in time is prior to the start of treatment for said subject, said second point in time is during the treatment period for said subject or after a treatment protocol/regime is completed by said subject and the level/amount of autoantibody is being determined relative to a baseline value and wherein the baseline value is the level/amount of autoantibody in a blood serum sample from the subject prior to the initiation of treatment for said neurodegenerative disease or condition.

9. The method of embodiment 7 or 8, wherein said assay measures the level/amount of autoantibody in a sample from a subject after development of a neurodegenerative disease or condition, said first point in time is prior to the development of said neurodegenerative disease or disorder, said second point in time is a point in time after said subject is diagnosed with said neurodegenerative disease or disorder, said assay measures the level/amount of autoantibody in said sample, the level/amount of autoantibody is determined relative to a baseline value and wherein the baseline value is the level/amount of autoantibody in blood serum samples from the subject prior to the development of said neurodegenerative disease or condition.

10. The method according to embodiments 1-9, wherein said biological sample is a blood sample.

11. The method according to embodiments 1-9, wherein said biological sample is a blood serum sample.

12. The method according to embodiments 1-9, wherein said biological sample is a CSF sample.

13. A method of treating a neurodegenerative disease or condition comprising administering a composition comprising a peptide fragment of SEQ ID NO: 1 to a subject having a neurodegenerative disease or condition, said peptide fragment comprising:

a) SEQ ID NO: 2, optionally fused to a heterologous sequence;

b) SEQ ID NO: 3, optionally fused to a heterologous sequence; or

c) a peptide fragment of SEQ ID NO: 1, 4, 5, 6, 7, 8, 9 or 10, optionally fused to a heterologous sequence.

14. The method according to embodiment 13, wherein said peptide fragment comprises SEQ ID NO: 2, optionally fused to a heterologous sequence.

15. The method according to embodiment 13, wherein said peptide fragment comprises SEQ ID NO: 3, optionally fused to a heterologous sequence.

16. The method according to embodiment 13, wherein said peptide fragment comprises a contiguous span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318 or 319 consecutive amino acids of SEQ ID NO: 1, 6, 8 or 9, wherein said contiguous span contains SEQ ID NO: 2, SEQ ID NO: 3, both SEQ ID NO: 2 and SEQ ID NO: 3 or a span of 5, 6, 7, 8, 9, 10 or 11 contiguous amino acids of SEQ ID NO: 2 and/or 3, said contiguous span optionally being fused to a heterologous sequence.

17. The method according to embodiments 13-16, wherein said neurodegenerative disease or condition is Huntington's disease, Parkinson's disease, post-traumatic stress disorder, stroke, spinal cord trauma, traumatic brain injury, multi-infarct dementia, epilepsy, amyotrophic lateral sclerosis, viral induced dementia, AIDS induced dementia, neurodegeneration associated with bacterial infection, brain ischemia, multiple sclerosis, Alzheimer's disease, senile dementia of the Alzheimer's type, mild cognitive impairment, age-related cognitive decline, corticobasal degeneration, dementia pugilistica, Down's syndrome, myotonic dystrophy, Niemann-Pick disease, Pick's disease, lower lateral sclerosis, paraneoplastic syndromes, encephalopathy, vascular dementia, primary progressive aphasia, diffuse Lewy body disease, progressive supranuclear palsy, Jacob Cruetzfeldt disease, Gerstmann Straussler disease, hydrocephalus, hereditary spinal paraplegia, myasthenia gravis, peripheral neuropathy, striatonigral degeneration, multi-system atrophy, familial tremor, Tourette's syndrome, myoclonus, Wilson's disease, Hallervorden-Spatz disease, neuroacanthocytosis, hemifacial spasm, Friedreich's ataxia, spinocerebellar ataxia, Sydenham's chorea, myositis or subacute sclerosing panencephalistis.

18. The method according to embodiments 1-17, wherein said subject has not been exposed to HTLV-1 virus or said subject does not have tropical spastic paraparesis.

19. The method according to embodiments 1-18, wherein said peptide fragment comprises a contiguous span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318 or 319 consecutive amino acids of SEQ ID NO: 1, 6, 8 or 9, wherein said contiguous span contains SEQ ID NO: 2, SEQ ID NO: 3, both SEQ ID NO: 2 and SEQ ID NO: 3 or a span of 5, 6, 7, 8, 9, 10 or 11 contiguous amino acids of SEQ ID NO: 2 and/or 3, said contiguous span optionally being fused to a heterologous sequence.

20. The method according to embodiments 1-18, wherein said peptide fragment comprises a contiguous span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313 or 314 consecutive amino acids of SEQ ID NO: 4, wherein said contiguous span contains SEQ ID NO: 2, SEQ ID NO: 3, both SEQ ID NO: 2 and SEQ ID NO: 3 or a span of 5, 6, 7, 8, 9, 10 or 11 contiguous amino acids of SEQ ID NO: 2 and/or 3, said contiguous span optionally being fused to a heterologous sequence.

21. The method according to embodiments 1-18, wherein said peptide fragment comprises a contiguous span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315 or 316 consecutive amino acids of SEQ ID NO: 5, wherein said contiguous span contains SEQ ID NO: 2, SEQ ID NO: 3, both SEQ ID NO: 2 and SEQ ID NO: 3 or a span of 5, 6, 7, 8, 9, 10 or 11 contiguous amino acids of SEQ ID NO: 2 and/or 3, said contiguous span optionally being fused to a heterologous sequence.

22. The method according to embodiments 1-18, wherein said peptide fragment comprises a contiguous span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319 or 320 consecutive amino acids of SEQ ID NO: 7, wherein said contiguous span contains SEQ ID NO: 2, SEQ ID NO: 3, both SEQ ID NO: 2 and SEQ ID NO: 3 or a span of 5, 6, 7, 8, 9, 10 or 11 contiguous amino acids of SEQ ID NO: 2 and/or 3, said contiguous span optionally being fused to a heterologous sequence.

23. The method according to embodiments 1-18, wherein said peptide fragment comprises a contiguous span of 5, 6, 7, 8, 9, 10, 11, 12, 13, 14, 15, 16, 17, 18, 19, 20, 21 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371 consecutive amino acids of SEQ ID NO: 10, wherein said contiguous span contains SEQ ID NO: 2, SEQ ID NO: 3, both SEQ ID NO: 2 and SEQ ID NO: 3 or a span of 5, 6, 7, 8, 9, 10 or 11 contiguous amino acids of SEQ ID NO: 2 and/or 3, said contiguous span optionally being fused to a heterologous sequence.

24. A kit comprising:

a) a polypeptide comprising SEQ ID NO: 1, 4, 5, 6, 7, 8, 9 or 10 or a peptide fragment of SEQ ID NO: 1, 4, 5, 6, 7, 8, 9 or 10, wherein said peptide fragment comprises SEQ ID NO: 2, SEQ ID NO: 3 or both SEQ ID NO: 2 and SEQ ID NO: 3; and

b) an detection antibody that specifically binds to human antibodies.

25. The kit according to embodiment 24, wherein said detection antibody is detectably labeled.

26. The kit according to embodiment 24, wherein said polypeptide is optionally fused to a heterologous sequence.

27. A device comprising a polypeptide comprising SEQ ID NO: 1, 4, 5, 6, 7, 8, 9 or 10 or a peptide fragment of SEQ ID NO: 1, 4, 5, 6, 7, 8, 9 or 10, wherein said peptide fragment comprises SEQ ID NO: 2, SEQ ID NO: 3 or both SEQ ID NO: 2 and SEQ ID NO: 3.

28. The device according to embodiment 27, wherein said polypeptide further comprises a heterologous sequence.

29. The device according to embodiment 27, wherein said device comprises a solid substrate to which said polypeptide comprising SEQ ID NO: 1, 4, 5, 6, 7, 8, 9 or 10 or a peptide fragment of SEQ ID NO: 1, 4, 5, 6, 7, 8, 9 or 10, wherein said peptide fragment comprises SEQ ID NO: 2, SEQ ID NO: 3 or both SEQ ID NO: 2 and SEQ ID NO: 3 is attached.

30. The device according to embodiment 29, wherein said solid substrate is a microtiter plate, a membrane, glass, nitrocellulose, plastic, polystyrene, a bead, or a dipstick.

We claim:

1. A method of detecting or diagnosing a neurodegenerative disease or condition in a subject comprising:
a) obtaining a biological sample from the subject; and
b) assaying the sample for the presence of at least one autoantibody that binds to SEQ ID NO: 1, 4, 5, 6, 7, 8, 9 or 10, and wherein presence of said at least one autoantibody indicates that the subject has, or is at risk of developing, a neurodegenerative disease or condition,
wherein said assaying comprises:
i) contacting a polypeptide comprising SEQ ID NO: 1, 4, 5, 6, 7, 8, 9 or 10 with said biological sample and detecting the binding of autoantibodies, wherein said biological sample is a blood serum sample; or
ii) contacting an epitope comprising a peptide fragment of SEQ ID NO: 1, 4, 5, 6, 7, 8, 9 or 10 with a biological sample and detecting the binding of autoantibodies to said polypeptide fragment, wherein said peptide fragment comprises SEQ ID NO: 2, SEQ ID NO: 3 or both SEQ ID NO: 2 and SEQ ID NO: 3.

2. The method according to claim 1, wherein the autoantibody binds an epitope comprising:
a) SEQ ID NO: 2, optionally fused to a heterologous sequence;
b) SEQ ID NO: 3, optionally fused to a heterologous sequence; or
c) a peptide fragment of SEQ ID NO: 1, 4, 5, 6, 7, 8, 9 or 10, wherein said peptide fragment is a contiguous span of amino acids that contains SEQ ID NO: 2; SEQ ID NO: 3; both SEQ ID NO: 2 and SEQ ID NO: 3; or a span of 5, 6, 7, 8, 9, 10 or 11 contiguous amino acids of SEQ ID NO: 2 and/or 3, said contiguous span optionally being fused to a heterologous sequence.

3. The method according to claim 1, wherein the subject has a neurodegenerative disease or condition selected from Huntington's disease, Parkinson's disease, post-traumatic stress disorder, stroke, spinal cord trauma, traumatic brain injury, multi-infarct dementia, epilepsy, amyotrophic lateral sclerosis, viral induced dementia, AIDS induced dementia, neurodegeneration associated with bacterial infection, brain ischemia, multiple sclerosis, Alzheimer's disease, senile dementia of the Alzheimer's type, mild cognitive impairment, age-related cognitive decline, corticobasal degeneration, dementia pugilistica, Down's syndrome, myotonic dystrophy, Niemann-Pick disease, Pick's disease, lower lateral sclerosis, paraneoplastic syndromes, encephalopathy, vascular dementia, primary progressive aphasia, diffuse Lewy body disease, progressive supranuclear palsy, Jacob Cruetzfeldt disease, Gerstmann Straussler disease, hydrocephalus, hereditary spinal paraplegia, myasthenia gravis, peripheral neuropathy, striatonigral degeneration, multi-system atrophy, familial tremor, Tourette's syndrome, myoclonus, Wilson's disease, Hallervorden-Spatz disease, neuroacanthocytosis, hemifacial spasm, Friedreich's ataxia, spinocerebellar ataxia, Sydenham's chorea, myositis or subacute sclerosing panencephalistis.

4. The method according to claim 3, wherein the subject has a neurodegenerative disease or condition selected from Parkinson's disease, post-traumatic stress disorder, multiple sclerosis, Alzheimer's disease or senile dementia of the Alzheimer's type.

5. The method according to claim 1, wherein the subject has a family history of at least one neurodegenerative disease or condition.

6. The method according to claim 1, wherein said assay measures the level/amount of autoantibody in said sample, the level/amount of autoantibody is determined relative to a baseline value and wherein the baseline value is an average or mean value of the level/amount of autoantibody in blood serum samples from a population of control subjects.

7. A method of monitoring the development or progress of a neurodegenerative disease or condition in a subject over a period of time comprising conducting an assay according to claim 1 at a first point in time and conducting said assay at a second point in time, said second point in time being later than said first point in time.

8. The method of claim 7, wherein said assay measures the level/amount of autoantibody in a sample from a subject after treatment for a neurodegenerative disease or condition, said first point in time is prior to the start of treatment for said subject, said second point in time is during the treatment period for said subject or after a treatment protocol/regime is completed by said subject and the level/amount of autoantibody is being determined relative to a baseline value and wherein the baseline value is the level/amount of autoantibody in a blood serum sample from the subject prior to the initiation of treatment for said neurodegenerative disease or condition.

9. The method of claim 7, wherein said assay measures the level/amount of autoantibody in a sample from a subject after development of a neurodegenerative disease or condition, said first point in time is prior to the development of said neurodegenerative disease or disorder, said second point in time is a point in time after said subject is diagnosed with said neurodegenerative disease or disorder, said assay measures the level/amount of autoantibody in said sample, the level/amount of autoantibody is determined relative to a baseline value and wherein the baseline value is the level/amount of autoantibody in blood serum samples from the subject prior to the development of said neurodegenerative disease or condition.

10. The method according to claim 1, wherein said biological sample is a blood sample.

11. The method according to claim 1, wherein said biological sample is a blood serum sample.

12. The method according to claim 1, wherein said biological sample is a CSF sample.

13. A method of treating a neurodegenerative disease or condition comprising administering a composition comprising a peptide fragment of SEQ ID NO: 1 to a subject having a neurodegenerative disease or condition, said peptide fragment comprising:
a) SEQ ID NO: 2, optionally fused to a heterologous sequence;
b) SEQ ID NO: 3, optionally fused to a heterologous sequence; or
c) a peptide fragment of SEQ ID NO: 1, 4, 5, 6, 7, 8, 9 or 10, optionally fused to a heterologous sequence.

14. The method according to claim 13, wherein said peptide fragment comprises SEQ ID NO: 2, optionally fused to a heterologous sequence.

15. The method according to claim 13, wherein said neurodegenerative disease or condition is Huntington's disease, Parkinson's disease, post-traumatic stress disorder, stroke, spinal cord trauma, traumatic brain injury, multi-infarct dementia, epilepsy, amyotrophic lateral sclerosis, viral induced dementia, AIDS induced dementia, neurodegeneration associated with bacterial infection, brain ischemia, multiple sclerosis, Alzheimer's disease, senile dementia of the Alzheimer's type, mild cognitive impairment, age-related cognitive decline, corticobasal degeneration, dementia pugilistica, Down's syndrome, myotonic dystrophy, Niemann-Pick disease, Pick's disease, lower lateral sclerosis, paraneoplastic syndromes, encephalopathy, vascular dementia, primary progressive aphasia, diffuse Lewy body disease, progressive supranuclear palsy, Jacob Cruetzfeldt disease, Gerstmann Straussler disease, hydrocephalus, hereditary spinal paraplegia, myasthenia gravis, peripheral neuropathy, striatonigral degeneration, multi-system atrophy, familial tremor, Tourette's syndrome, myoclonus, Wilson's disease, Hallervorden-Spatz disease, neuroacanthocytosis, hemifacial spasm, Friedreich's ataxia, spinocerebellar ataxia, Sydenham's chorea, myositis or subacute sclerosing panencephalistis.

16. The method according to claim 1, wherein said subject has not been exposed to HTLV-1 virus or said subject does not have tropical spastic paraparesis.

17. A device comprising a polypeptide comprising SEQ ID NO: 1, 4, 5, 6, 7, 8, 9 or 10 or a peptide fragment of SEQ ID NO: 1, 4, 5, 6, 7, 8, 9 or 10, wherein said peptide fragment comprises SEQ ID NO: 2, SEQ ID NO: 3 or both SEQ ID NO: 2 and SEQ ID NO: 3.

18. The device according to claim 17, wherein said polypeptide further comprises a heterologous sequence.

19. The device according to claim 17, wherein said device comprises a solid substrate to which said polypeptide comprising SEQ ID NO: 1, 4, 5, 6, 7, 8, 9 or 10 or a peptide fragment of SEQ ID NO: 1, 4, 5, 6, 7, 8, 9 or 10, wherein said peptide fragment comprises SEQ ID NO: 2, SEQ ID NO: 3 or both SEQ ID NO: 2 and SEQ ID NO: 3 is attached.

20. The device according to claim 19, wherein said solid substrate is a microtiter plate, a membrane, glass, nitrocellulose, plastic, polystyrene, a bead, or a dipstick.
